(19) United States
(12) Patent Application Publication
(10) Pub. No.: US 2009/0317861 A1
(43) Pub. Date: Dec. 24, 2009

(54) CELL-FREE SYNTHESIS OF VIRUS LIKE PARTICLES

(76) Inventors: Bradley C. Bundy, Mountain View, CA (US); James Robert Swartz, Menlo Park, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/305,616

(22) PCT Filed: Jun. 29, 2007
(86) PCT No.: PCT/US07/15270

§ 371 Date: Jul. 2, 2009

Related U.S. Application Data
(60) Provisional application No. 60/817,909, filed on Jun. 29, 2006.

(51) Int. Cl. C12P 21/00

(52) U.S. Cl. 435/68.1

(57) ABSTRACT

Methods are provided for the utilization of bacterial cell-free extracts in the synthesis of high yields of virus like particles.

### BACKGROUND OF THE INVENTION

Protein synthesis is a fundamental biological process that underlies the development of polypeptide therapeutics, diagnostics, and industrial enzymes. With the advent of recombinant DNA (rDNA) technology, it has become possible to harness the catalytic machinery of the cell to produce a desired protein. This can be achieved within the cellular environment or in vitro using extracts derived from cells.

Cell-free protein synthesis offers several advantages over in vivo protein expression methods. Cell-free systems can direct most, if not all, of the metabolic resources of the cell towards the exclusive production of one protein. Moreover, the lack of a cell wall in vitro is advantageous since it allows for control of the synthesis environment. For example, tRNA levels can be changed to reflect the codon usage of genes being expressed. The redox potential, pH, or ionic strength can also be altered with greater flexibility than in vivo since we are not concerned about cell growth or viability. Furthermore, direct recovery of purified, properly folded protein products can be easily achieved.

In vitro translation is also recognized for its ability to incorporate unnatural and isotope-labeled amino acids as well as its capability to produce proteins that are unstable, insoluble, or cytotoxic in vivo. In addition, cell-free protein synthesis may play a role in revolutionizing protein engineering and proteomic screening technologies. The cell-free method bypasses the laborious processes required for cloning and transforming cells for the expression of new gene products in vivo, and is becoming a platform technology for this field.

Virus-like particles (VLPs), formed from structural proteins of viruses, have received considerable attention for vaccines, targeted drug delivery, targeted gene delivery, and nanotechnology applications. The vast majority of eukaryote-infecting-virus-based VLPs have been synthesized using the insect-cell-based baculovirus expression system or mammalian-cell-based protein expression systems. Although the synthesis of virus-like particles has been attempted in cell-free systems, yields have been extremely low in eukaryotic cell-free systems (Lingappa et al. 2005. Virology 333:114), and assembly has failed in conventional prokaryotic systems (Katanaev et al. 1996. FEBS 397:143).

Nonetheless, technology providing VLP synthesis in prokaryote-based cell-free systems is of great interest because of the potential advantages mentioned above.

### Relevant Literature

U.S. Pat. No. 6,337,191 B1; Swartz et al. U.S. Patent Published Application 20040209321; Swartz et al., International Published Application WO 2004/016778; Swartz et al. U.S. Patent Published Application 2005-0054032-A1; Swartz et al. U.S. Patent Published Application 2005-0054044-A1; Swartz et al International Published Application WO 2005/052117. Calhoun and Swartz (2005) Biotechnol Bioeng 90(5):606-133; Jewett and Swartz (2004) Biotechnol Bioeng 86(1):19-26; Jewett et al. (2002) Prokaryotic Systems for In Vitro Expression. In: Weiner M, Lu Q, editors. Gene cloning and expression technologies. Westborough, Mass.: Eaton Publishing. p 391-411; Lin et al. (2005) Biotechnol Bioeng 89(2): 148-56.

U.S. Pat. No. 6,593,103, Lingappa et al. Noad and Roy (2003). Virus-like particles as immunogens. Trends in Microbiology 11(9):438-444. Palucha et al. 2005. Virus-like particles: models for assembly studies and foreign epitope carriers. Progress in Nucleic Acid Research and Molecular Biology 80:135-168. Spirin et al. 1988. A continuous cell-free translation system capable of producing polypeptides in high yield. Science 242:1162-1164.

### SUMMARY OF THE INVENTION

Methods are provided for high yield cell-free synthesis of stable virus like particles. In the methods of the invention, a prokaryotic cell-free synthesis reaction is used to produce at least one viral coat protein, which self-assembles into a stable virus like particle, or capsid. The synthesis may be performed as a coupled transcription and translation reaction, in a reaction mix substantially free of polyethylene glycol. These methods provide for high yields of assembled nanoparticles.

In one embodiment, the synthesis reaction conditions provide for in vitro activation of oxidative phosphorylation. The activation of oxidative phosphorylation may be evidenced by sensitivity of synthesis to electron transport chain inhibitors.

### BRIEF DESCRIPTION OF THE DRAWINGS

FIG. 1: MS2 coat protein gene nucleotide sequence optimized for PCR synthesis from commercial oligonucleotides and for expression by E. coli cell extracts. This gene was inserted using the underlined NdeI and SalI sites into commercial vector pET24a (Novagen, USA) to produce pET24a-MS2 cp.

FIG. 2: Cell-free protein synthesis yield using pET24a-MS2 cp expression vector (30 ul reactions, n=5).

FIG. 3A: Analysis of MS2 coat protein (13.7 kD) production using SDS-Page gel analysis (10% Bis-Tris Gel w/MOPS running buffer, Invitrogen; 60 min at 60 mA running conditions; Commassie Blue Stain, BioRad). Lane M: Mark12 Standard (Invitrogen); Lane 1: 5 ul MS2 CFPS post-reaction; Lane 2: 5 ul MS2 CFPS after dialysis; Lane 3: 5 ul CFPS post reaction

FIG. 3B: Autoradiogram of gel shown in FIG. 4A. Lanes 1R through 3R represent lanes 1 through 3.

FIG. 4: The 10%-40% (with 2.5% steps) sucrose density gradient velocity sedimentation profile without EDTA. The ribosome profile is shown by the 254 nm absorbance profile (solid). The location of radiolabeled MS2 coat protein is determined by scintillation counting of incorporated ¹⁴C-leucine (dashed).

FIG. 5A: SDS-Page Gel (10% Bis-Tris Gel w/MOPS running buffer, Invitrogen; 60 min at 60 mA running conditions; Commassie Blue Stain, BioRad) of fractions 11 through 17 of the sucrose density gradient profile shown in FIG. 4 (25 JAI of fraction, 10.9 μl NuPAGE LDS Sample Buffer-Invitrogen, 0.625 mM DTT-Invitrogen). Lane: (M) Mark12 Standard (Invitrogen), (1) fraction #11, (2) fraction #12, (3) fraction #13, (4) fraction #14, (5) fraction #15, (6) fraction #16, (7) fraction #17.

FIG. 5B: Autoradiogram of gel shown in FIG. 4. Lanes 1R through 7R represent lanes 1 through 7.

FIG. 6A-C: Transmission Electron Microscopy (JEOL TEM 1230 with Gatan 967 CCD camera) images of concentrated sucrose gradient fractions of MS2 VLP at 120k, 200k, and 500k× magnification. Scale bar in bottom left corner represents 200 nm (A), 110 nm (B), and 50 nm (C).

FIG. 7: 10%-40% (with 2.5% steps) sucrose density gradient velocity sedimentation profile with 15 mM EDTA. The location of radiolabeled MS2 coat protein in μg/mL is shown as determined by scintillation counting of incorporated ¹⁴C-leucine (dashed). The decrease in ribosome complex concentration is shown by the 280 nm absorbance profile (solid).

FIG. 8: 10%-40% (with 5% steps) sucrose density gradient velocity sedimentation profiles after VLP incubation for 1 hr at various pHs as determined by scintillation counting of incorporated ¹⁴C-leucine.

### DETAILED DESCRIPTION OF THE EMBODIMENTS

High yield production of virus like particles (VLP) is accomplished by synthesis in a prokaryotic cell-free reaction. The synthesis may be accomplished in coupled transcription and translation reactions where the virus coat protein gene may be provided in a suitable vector, e.g. plasmid, etc., operably linked to a promoter active in the transcription system. The virus protein of interest is synthesized in a reaction mixture that allows self-assembly of the capsid structure, e.g. a reaction mixture substantially free of polyethylene glycol. In some embodiments, the VLP is assembled from a single coat protein. In other embodiments, the VLP is assembled from two, three or more coat proteins. Bacteriophage VLP are of particular interest. The VLPs find use an immunogens, carrier particles, etc.

The methods of the invention provide for high yields of active, i.e. self-assembling, protein. In some embodiments, the yield of active virus coat protein is at least about 50 μg/ml of reaction mixture; at least about 100 μg/ml of reaction mixture; at least about 250 μg/ml, at least about 400 μg/ml of reaction mixture; or more. A substantial portion of the coat protein is assembled into stable VLPs, usually at least about 25%, at least about 50%, at least about 75% or more.

Cell-free protein synthesis provides several advantages over in vivo protein synthesis methods for producing VLPs. In cell-free systems most of the metabolic resources available can be directed toward the exclusive production of the desired protein(s). The cell-free system allows greater control of the transcription/translation environment and ease of VLP recovery and purification, as the system lacks a cell-wall and membrane components. For example, the redox potential, pH, and/or ionic strength can be altered which is necessary for optimum assembly, disassembly, and reassembly of many VLPs. In many cases the VLPs must be disassembled and reassembled in vitro to purge any biomaterial encapsulated during the assembly process and assemble a higher concentration of structural proteins. By using the cell-free transcription-translation system, the VLP production process is streamlined by eliminating the in vivo production step.

Of particular interest is the ability of cell-free protein synthesis to produce at high expression levels the virus structural proteins that are often cytotoxic in vivo. By employing the recently developed linear DNA template cell-free technology, the time and labor intensive process of cloning and transforming an adequate expression vector is greatly reduced. Thus, the production of a VLP-based vaccines could be expedited for cancer treatment or viral epidemics.

The methods of the present invention provide for virus like particles that have biological activity comparable to the native assemblies. One may determine the specific activity of a protein in a composition by determining the level of activity in a functional assay, quantitating the amount of protein present in a non-functional assay, e.g. immunostaining, ELISA, quantitation on coomassie or silver stained gel, etc., and determining the ratio of biologically active protein to total protein. Generally, the specific activity as thus defined will be at least about 5% that of the native protein, usually at least about 10% that of the native protein, and may be about 25%, about 50%, about 90% or greater.

In some embodiments, the cell-free reaction mixture will have activation of oxidative phosphorylation as obtained by a combination of reaction conditions, which conditions may include, without limitation, the use of biological extracts derived from bacteria grown on a glucose containing medium; an absence of polyethylene glycol; and optimized magnesium concentration. The system does not require the addition of commonly used secondary energy sources, which energy sources typically contain high energy phosphate bonds, such as phosphoenolpyruvate, creatine phosphate, acetyl phosphate, glucose-6-phosphate, pyruvate or glycolytic intermediates. The reaction may be further improved by employing ions and compounds in the cell-free reaction mixture that are commonly found in the E. coli cytoplasm.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Virus like particle. As used herein, the term “virus like particle” refers to a stable macromolecular assembly of one or more virus proteins, usually viral coat proteins. The number of separate protein chains in a VLP will usually be at least about 60 proteins, about 80 proteins, at least about 120 proteins, or more, depending on the specific viral geometry. In the methods of the invention, the cell-free synthesis reaction mixture provides conditions permissive for self-assembly into the capsid structure, even where the concentration of coat proteins may be dilute relative to the concentrations associated with in vivo viral synthesis, e.g. less than about 500 μg/ml, less than about 400 μg/ml, less than about 250 μg/ml. The methods of the invention provide for synthesis of the coat protein in the absence of the virus polynucleotide genome, and thus the capsid may be empty, or contain non-viral components, e.g. mRNA fragments, etc. The cell-free synthesis reaction mixtures of the present invention surprisingly provide conditions permissive for self-assembly of coat proteins into a capsid structure displaying helical or icosahedral symmetry.

A stable VLP maintains the association of proteins in a capsid structure under physiological conditions for extended periods of time, e.g. for at least about 24 hrs, at least about 1 week, at least about 1 month, or more. Once assembled, the VLP can have a stability commensurate with the native virus particle, e.g. upon exposure to pH changes, heat, freezing, ionic changes, etc. Additional components of VLPs, as known in the art, can be included within or disposed on the VLP. VLPs do not contain intact viral nucleic acids, and they are non-infectious. In some embodiments there is sufficient viral surface envelope glycoprotein and/or adjuvant molecules on the surface of the VLP so that when a VLP preparation is formulated into an immunogenic composition and administered to an animal or human, an immune response (cell-mediated or humoral) is raised.

Viruses can be classified into those with helical symmetry or icosahedral symmetry. Generally recognized capsid morphologies include: icosahedral (including icosahedral proper, isometric, quasi-isometric, and geminate or “twinned”), polyhedral (including spherical, ovoid, and lemon-shaped), bacilliform (including rhabdo- or bullet-shaped, and fusiform or cigar-shaped), and helical (including rod, cylindrical, and filamentous); any of which may be tailed and/or may contain surface projections, such as spikes or knobs.

In one embodiment of the invention, the coat protein is selected from the capsids of viruses classified as having any icosahedral morphology, and the VLP has an icosahedral geometry. Generally, viral capsids of icosahedral viruses are composed of numerous protein sub-units arranged in icosahedral (cubic) symmetry. Native icosahedral capsids can be built up, for example, with 3 subunits forming each triangular face of a capsid, resulting in 60 subunits forming a complete capsid. A representative of this small viral structure is bacteriophage ØX174. Many icosahedral virus capsids contain more than 60 subunits. Many capsids of icosahedral viruses contain an antiparallel, eight-stranded beta-barrel folding motif. The motif has a wedge-shaped block with four beta strands (designated BIDG) on one side and four (designated CHEF) on the other. There are also two conserved alpha-helices (designated A and B), one is between betaC and betaD, the other between betaE and betaF.

Virus coat proteins of interest include any of the known virus type, e.g. dsDNA viruses, such as smallpox (variola); vaccinia; herpesviruses including varicella-zoster; HSV1, HSV2, KSVH, CMV, EBV; adenovirus; hepatitis B virus; SV40; T even phages such as T4 phage, T2 phage; lambda phage; etc. Single stranded DNA viruses include phiX-174; adeno-associated virus, etc. Negative-stranded RNA viruses include measles virus; mumps virus; respiratory syncytial virus (RSV); parainfluenza viruses (PIV); metapneumovirus; rabies virus; Ebola virus; influenza virus; etc. Positive-stranded RNA viruses include polioviruses; rhinoviruses; coronaviruses; rubella; yellow fever virus; West Nile virus; dengue fever viruses; equine encephalitis viruses; hepatitis A and hepatitis C viruses; tobacco mosaic virus (TMV); etc. Double-stranded RNA viruses include reovirus; etc. Retroviruses include rous sarcoma virus; lentiviruses such as HIV-1 and HIV-2; etc.

Bacteriophages are of interest, e.g. the MS2 bacteriophage. Myoviridae (phages with contractile tails) include mu-like viruses; P1-like viruses, e.g. P1; phiW39, etc.; P2-like viruses; SPO-1-like viruses; T4-like viruses; etc. Podoviridae (phages with short tails) include N4-like viruses; P22-like viruses, e.g. P22; phi-29-like viruses, e.g. phi-29; T7-like viruses, e.g. T3; T7; W31; etc. Siphoviridae (phages with long non-contractile tails) include c2-like viruses; L5-like viruses; Lambda-like viruses, e.g. phage lambda, HK022; HK97, etc.; N15-like viruses; PhiC31-like viruses; psiM1-like viruses; T1-like viruses, e.g. phage T1, etc. Microviridae (isometric ssDNA phages) include Chlamydiamicrovirus; Microvirus, e.g. phage alpha 3, phage WA13, etc.; phage G4; phage phiX174 and related coliphages. Many additional phages known to those of skill in the art remain unclassified. The sequence of many coat proteins are publicly available.

The nucleic acid sequence encoding the viral capsid or proteins can be modified to alter the formation of VLPs (see e.g. Brumfield, et al. (2004) J. Gen. Virol. 85: 1049-1053). For example, three general classes of modification are most typically generated for modifying VLP expression and assembly. These modifications are designed to alter the interior, exterior or the interface between adjacent subunits in the assembled protein cage. To accomplish this, mutagenic primers can be used to: (i) alter the interior surface charge of the viral nucleic acid binding region by replacing basic residues (e.g. K, R) in the N terminus with acidic glutamic acids (Douglas et al., 2002b); (ii) delete interior residues from the N terminus (in CCMV, usually residues 4-37); (iii) insert a cDNA encoding an 11 amino acid peptide cell-targeting sequence (Graf et al., 1987) into a surface exposed loop and (iv) modify interactions between viral subunits by altering the metal binding sites (in CCMV, residues 81/148 mutant).

In vitro synthesis, as used herein, refers to the cell-free synthesis of polypeptides in a reaction mix comprising biological extracts and/or defined reagents. The reaction mix will comprise a template for production of the macromolecule, e.g. DNA, mRNA, etc.; monomers for the macromolecule to be synthesized, e.g. amino acids, nucleotides, etc., and such co-factors, enzymes and other reagents that are necessary for the synthesis, e.g. ribosomes, tRNA, polymerases, transcriptional factors, etc. Such synthetic reaction systems are well-known in the art, and have been described in the literature. The cell free synthesis reaction may be performed as batch, continuous flow, or semi-continuous flow, as known in the art.

In some embodiments of the invention, cell free synthesis is performed in a reaction where oxidative phosphorylation is activated, e.g. the CYTOMIM™ system. The activation of the respiratory chain and oxidative phosphorylation is evidenced by an increase of polypeptide synthesis in the presence of O₂. In reactions where oxidative phosphorylation is activated, the overall polypeptide synthesis in presence of O₂ is reduced by at least about 40% in the presence of a specific electron transport chain inhibitor, such as HQNO, or in the absence of O₂. The reaction chemistry may be as described in international patent application WO 2004/016778, herein incorporated by reference.

The CYTOMIM™ environment for synthesis utilizes cell extracts derived from bacterial cells grown in medium containing glucose and phosphate, where the glucose is present initially at a concentration of at least about 0.25% (weight/volume), more usually at least about 1%; and usually not more than about 4%, more usually not more than about 2%. An example of such media is 2YTPG medium. However one of skill in the art will appreciate that many culture media can be adapted for this purpose, as there are many published media suitable for the growth of bacteria such as E. coli, using both defined and undefined sources of nutrients (see Sambrook, J., E. F. Fritsch, and T. Maniatis. 1989. Molecular Cloning: A Laboratory Manual, 2^{nd} edition. Cold Spring Harbor University Press, Cold Spring Harbor, N.Y. for examples of glucose containing media). Alternatively, the culture may be grown using a protocol in which the glucose is continually fed as required to maintain a high growth rate in either a defined or complex growth medium.

The reaction mixture may be supplemented by the inclusion of vesicles, e.g. an inner membrane vesicle solution. Where provided, such vesicles may comprise from about 0 to about 0.5 volumes, usually from about 0.1 to about 0.4 volumes.

In the reaction mix PEG will usually be present in not more than trace amounts, for example less than 0.1%, and may be less than 0.01%. Reactions that are substantially free of PEG contain sufficiently low levels of PEG that, for example, oxidative phosphorylation is not PEG-inhibited, and the self-assembly of virus-like particles is not inhibited. The molecules spermidine and putrescine may be used in the place of PEG. Spermine or spermidine is present at a concentration of at least about 0.5 mM, usually at least about 1 mM, preferably about 1.5 mM, and not more than about 2.5 mM. Putrescine is present at a concentration of at least about 0.5 mM, preferably at least about 1 mM, preferably about 1.5 mM, and not more than about 2.5 mM. The spermidine and/or putrescine may be present in the initial cell extract or may be separately added.

The concentration of magnesium in the reaction mixture affects the overall synthesis. Often there is magnesium present in the cell extracts, which may then be adjusted with additional magnesium to optimize the concentration. Sources of magnesium salts useful in such methods are known in the art. In one embodiment of the invention, the source of magnesium is magnesium glutamate. A preferred concentration of magnesium is at least about 5 mM, usually at least about 10 mM, and preferably a least about 12 mM; and at a concentration of not more than about 25 mM, usually not more than about 20 mM. Other changes that may enhance synthesis include the omission of HEPES buffer and phosphoenol pyruvate from the reaction mixture.

The system can be run under aerobic and anaerobic conditions. Oxygen may be supplied, particularly for reactions larger than 15 μl, in order to increase synthesis yields. The headspace of the reaction chamber can be filled with oxygen; oxygen may be infused into the reaction mixture; etc. Oxygen can be supplied continuously or the headspace of the reaction chamber can be refilled during the course of protein expression for longer reaction times. Other electron acceptors, such as nitrate, sulfate, or fumarate may also be supplied in conjunction with preparing cell extracts so that the required enzymes are active in the cell extract.

It is not necessary to add exogenous cofactors for activation of oxidative phosphorylation. Compounds such as nicotinamide adenine dinucleotide (NADH), NAD⁺, or acetyl-coenzyme A may be used to supplement protein synthesis yields but are not required. Addition of oxalic acid, a metabolic inhibitor of phosphoenolpyruvate synthetase (Pps), may be beneficial in increasing protein yields, but is not necessary.

The template for cell-free protein synthesis can be either mRNA or DNA, preferably a combined system continuously generates mRNA from a DNA template with a recognizable promoter. Either endogenous RNA polymerase is used, or an exogenous phage RNA polymerase, typically T7 or SP6, is added directly to the reaction mixture. Alternatively, mRNA can be continually amplified by inserting the message into a template for QB replicase, an RNA dependent RNA polymerase. Purified mRNA is generally stabilized by chemical modification before it is added to the reaction mixture. Nucleases can be removed from extracts to help stabilize mRNA levels. The template can encode for any particular gene of interest.

Other salts, particularly those that are biologically relevant, such as manganese, may also be added. Potassium is generally present at a concentration of at least about 50 mM, and not more than about 250 mM. Ammonium may be present, usually at a concentration of not more than 200 mM, more usually at a concentration of not more than about 100 mM. Usually, the reaction is maintained in the range of about pH 5-10 and a temperature of about 20°-50° C.; more usually, in the range of about pH 6-9 and a temperature of about 25°-40° C. These ranges may be extended for specific conditions of interest.

Metabolic inhibitors to undesirable enzymatic activity may be added to the reaction mixture. Alternatively, enzymes or factors that are responsible for undesirable activity may be removed directly from the extract or the gene encoding the undesirable enzyme may be inactivated or deleted from the chromosome.

Biological extracts. For the purposes of this invention, biological extracts are any preparation comprising the components of protein synthesis machinery, usually a bacterial cell extract, wherein such components are capable of expressing a nucleic acid encoding a desired protein. Thus, a bacterial extract comprises components that are capable of translating messenger ribonucleic acid (mRNA) encoding a desired protein, and optionally comprises components that are capable of transcribing DNA encoding a desired protein. Such components include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N¹⁰-formyltetrahydrofolate, formylmethionine-tRNAf^{Met} synthetase, peptidyl transferase, initiation factors such as IF-1, IF-2 and IF-3, elongation factors such as EF-Tu, EF-Ts, and EF-G, release factors such as RF-1, RF-2, and RF-3, and the like.

In a preferred embodiment of the invention, the reaction mixture comprises extracts from bacterial cells, e.g. E. coli S30 extracts, as is known in the art. For convenience, the organism used as a source of extracts may be referred to as the source organism. Methods for producing active extracts are known in the art, for example they may be found in Pratt (1984), Coupled transcription-translation in prokaryotic cell-free systems, p. 179-209, in Hames, B. D. and Higgins, S. J. (ed.), Transcription and Translation: A Practical Approach, IRL Press, New York. Kudlicki et al. (1992) Anal Biochem 206(2):389-93 modify the S30 E. coli cell-free extract by collecting the ribosome fraction from the S30 by ultracentrifugation. While such extracts are a useful source of ribosomes and other factors necessary for protein synthesis, they can also contain small amounts of enzymes responsible for undesirable side-reactions that are unrelated to protein synthesis, but which modulate the oxidizing environment of the reaction, and which can act to reduce the groups on the nascent polypeptide and the redox buffer.

Vesicles are optionally added to the reaction mix. Vesicles may purified from the organism from which the extract is derived (see Muller and Blobel (1984) “In vitro translocation of bacterial proteins across the plasma membrane of Escherichia coli”, PNAS 81:7421-7425); or isolated from any other suitable cell, e.g. mammalian cells including cells from the species of target protein; or synthetic. Vesicles are typically added at a concentration of 0.1 to 5 mg/ml lipids, more preferably about 0.4 to 2.5 mg/ml. Vesicles may be purified by sucrose density gradient centrifugation or by other means known in the art. Vesicle preparation methods include, without limitation: homogenization, French press, extrusion, freeze/thaw, sonication, osmotic lysis, lysozyme/EDTA treatment, and the like. Other components that affect membrane protein insertion or folding may be added to the cell-free reaction mixture, including SRP, Ffh, 4.5S RNA, FtsY, and SecA. Also, other components may be added to the reaction such as specific enzymes and their substrates as required for capsid protein modification and capsid assembly.

### Methods for Synthesis

The reactions may utilize a large scale reactor, small scale, or may be multiplexed to perform a plurality of simultaneous syntheses. Continuous reactions will use a feed mechanism to introduce a flow of reagents, and may isolate the end-product as part of the process. Batch systems are also of interest, where additional reagents may be introduced to prolong the period of time for active synthesis. A reactor may be run in any mode such as batch, extended batch, semi-batch, semi-continuous, fed-batch and continuous, and which will be selected in accordance with the application purpose.

The reactions may be of any volume, either in a small scale, usually at least about 1 μl and not more than about 15 μl, or in a scaled up reaction, where the reaction volume is at least about 15 μl, usually at least about 50 μl, more usually at least about 100 μl, and may be 500 μl, 1000 μl, or greater. In most cases, individual reactions will not be more than about 10 ml, although multiple reactions can be run in parallel. However, in principle, reactions may be conducted at any scale as long as sufficient oxygen (or other electron acceptor) is supplied when needed.

In addition to the above components such as cell-free extract, genetic template, and amino acids, materials specifically required for protein synthesis may be added to the reaction. These materials include salts, folinic acid, cyclic AMP, inhibitors for protein or nucleic acid degrading enzymes, inhibitors or regulators of protein synthesis, adjusters of oxidation/reduction potential(s), non-denaturing surfactants, buffer components, spermine, spermidine, putrescine, etc.

The salts preferably include potassium, magnesium, and ammonium salts (e.g. of acetic acid or glutamic acid). One or more of such salts may have an alternative amino acid as a counter anion. There is an interdependence among ionic species for optimal concentration. These ionic species are typically optimized with regard to protein production. When changing the concentration of a particular component of the reaction medium, that of another component may be changed accordingly. For example, the concentrations of several components such as nucleotides and energy source compounds may be simultaneously adjusted in accordance with the change in those of other components. Also, the concentration levels of components in the reactor may be varied over time. The adjuster of oxidation/reduction potential may be dithiothreitol, ascorbic acid, glutathione, cysteine, and/or their oxidized forms.

In a semi-continuous operation mode, the outside or outer surface of the membrane is put into contact with predetermined solutions that are cyclically changed in a predetermined order. These solutions contain substrates such as amino acids and nucleotides. At this time, the reactor is operated in dialysis, diafiltration batch or fed-batch mode. A feed solution may be supplied to the reactor through the same membrane or a separate injection unit. Synthesized protein and particles are accumulated in the reactor, and then are isolated and purified according to the usual method for protein purification after completion of the system operation. Product may also be continuously isolated, for example by affinity adsorption from the reaction mixture either in situ or in a circulation loop as the reaction fluid is pumped past the adsorption matrix.

Where there is a flow of reagents, the direction of liquid flow can be perpendicular and/or tangential to a membrane. Tangential flow is effective for recycling ATP and for preventing membrane plugging and may be superimposed on perpendicular flow. Flow perpendicular to the membrane may be caused or effected by a positive pressure pump or a vacuum suction pump or by applying transmembrane pressure using other methods known in the art. The solution in contact with the outside surface of the membrane may be cyclically changed, and may be in a steady tangential flow with respect to the membrane. The reactor may be stirred internally or externally by proper agitation means.

During protein synthesis in the reactor, the protein isolating means for selectively isolating the desired protein or particle may include a unit packed with particles coated with antibody molecules or other molecules immobilized with a component for adsorbing the synthesized, desired product. Preferably, the product isolating means comprises two columns for alternating use.

The amount of protein produced in a translation reaction can be measured in various fashions. One method relies on the availability of an assay which measures the activity of the particular protein being translated. An example of an assay for measuring protein activity is a luciferase assay system, or chloramphenical acetyl transferase assay system. These assays measure the amount of functionally active protein produced from the translation reaction. Activity assays will not measure full length protein that is inactive due to improper protein folding or lack of other post translational modifications necessary for protein activity. Particle assembly may similarly be measured by methods known in the art; for example, by the use of sucrose density centrifugation analysis.

Another method of measuring the amount of protein produced in coupled in vitro transcription and translation reactions is to perform the reactions using a known quantity of radiolabeled amino acid such as ³⁵S-methionine, ³H-leucine or ¹⁴C-leucine and subsequently measuring the amount of radiolabeled amino acid incorporated into the newly translated protein. Incorporation assays will measure the amount of radiolabeled amino acids in all proteins produced in an in vitro translation reaction including truncated protein products. The radiolabeled protein may be further separated on a protein gel, and by autoradiography confirmed that the product is the proper size and that secondary protein products have not been produced.

Kits for the practice of the subject methods may also be provided. Such kits may include bacterial extracts for protein synthesis, buffers appropriate for reactions where oxidative phosphorylation is activated, and suitable vectors.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

### Synthesis of Optimized MS2 Gene

### Materials and Methods

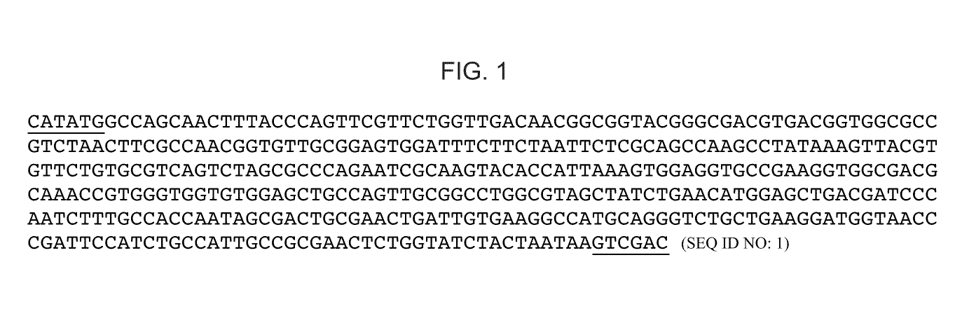

Plasmid Construction. The MS2 Coat Protein gene was optimized for both E. coli tRNA relative concentrations (preferred codons) and synthesis from oligonucleotides using DNAworks (Hoover D and Lubkowski J, 2002 Nucleic Acids Res 30(10):e43). Oligonucleotides (60 bp average length, Operon Technologies, USA) based on sequences recommended by DNAworks were assembled into the optimized MS2 coat protein gene nucleotide sequence using two-step PCR. pET24a-MS2 cp was generated by ligation (T4 DNA ligase, NEB, USA) of the optimized MS2 coat protein sequence into the pET-24a(+) vector (Novagen, USA) at the NdeI and SalI restriction sites. pET24a-MS2 cp was transformed into DH5α cells (One Shot MAXX Efficiency DH5α-T1^{R} Competent Cells, Invitrogen) and the plasmid was purified with Qiagen Plasmid Maxi Kit (Qiagen, Valencia, Calif.) for use in cell-free protein synthesis. See FIG. 1 for sequence

### Example 2

### Expression of MS2 Coat Protein

### Materials and Methods:

PANOx SP Cell-free Expression System. The PANOx SP system (described in Jewett and Swartz, 2004 Biotechnol Bioeng 86(1):1926) cell-free reactions were 30 μl in volume and were incubated at 37° C. for 3 hr in 1.5 ml eppendorf tubes. The reaction includes the following components: 1.2 mM ATP, 0.85 mM each of GTP, UTP, and CTP, 34 μg/mL folinic acid, 170.6 μg/mL E. coli tRNA mixture, 24 nM plasmid, 100 μg/mLT7 RNA polymerase, 5 μM I-[U-14C] leucine, 2 mM each of 20 unlabeled amino acids, 0.33 mM nicotinamide adenine dinucleotide (NAD), 0.27 mM coenzyme A (CoA), 30 mM phosphoenolpyruvate, 1.5 mM spermidine, 1 mM putrescine, 170 mM potassium glutamate, 10 mM ammonium glutamate, 20 mM magnesium glutamate, 2.7 mM sodium oxalate, and 24% v/v of S30 extract prepared as described (Liu et al., 2005 Biotechnol Prog 21:460-465).

Protein Yield Determination. Total synthesized protein yields were determined by TCA-precipitation and radioactivity measurements in a liquid scintillation counter (LS3801, Beckman Coulter, Inc.). Soluble yields were determined by TCA-precipitation and scintillation counting of the supernatants following sample centrifugation at 25° C. and 15,000 RCF for 15 min. The detailed procedure is described by Jewett and Swartz (Jewett M C and Swartz J R, 2004 Biotechnol Prog 20:102-109).

SDS-PAGE and Autoradiography. Five μl of sample was applied to a NuPAGE 10% Bis-Tris Gel (Invitrogen, La Jolla, Calif.) with Mark12 MW Standard (Invitrogen) molecular weight markers. Samples were run under reduced conditions in the presence of 62.5 mM dithiothreitol (DTT) (Invitrogen) and 7.25 μl NuPAGE LDS Sample Buffer (InVitrogen). Gels were stained with Commassie Blue Stain (BioRad) and dried with a gel dryer, model 583 (Bio-Rad, Richmond, Calif.) before being exposed to Kodak scientific imaging films (Rochester, N.Y.). Soluble proteins were separated from insoluble fractions by centrifugation at 16,000 g for 15 min.

### Results

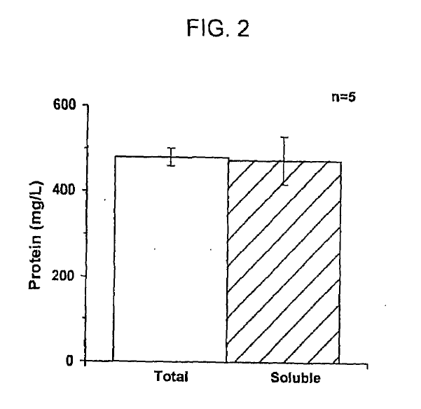

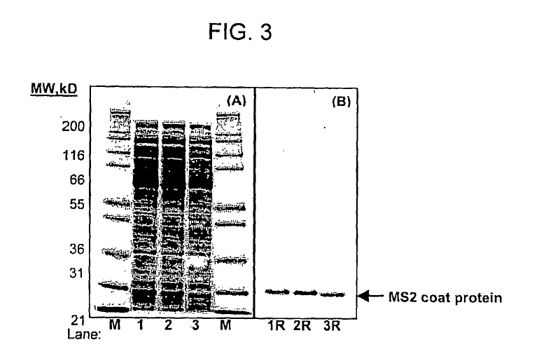

Five 30 μl PANOx SP system cell-free reactions using pET24a-MS2 cp were performed on two different days (2 reactions one day and 3 reactions the next day). The total and soluble yields were determined to be 479 μg/ml (±16 μg/ml) and 473 μg/ml (±44 μg/ml) respectively as indicated in FIG. 2. An SDS-PAGE gel was run using 5 μl of PANOx SP system cell-free reaction product (with pET24a-MS2 cp vector) with total and soluble fractions followed by autoradiography with results shown in FIG. 3.

### Example 3

### Demonstrating Assembly of MS2 Capsid

Dialysis. To remove unincorporated L-[U-¹⁴C] leucine, the cell-free produced was dialyzed in 6-8000 MWCO Specra/Pro Molecular porous Membrane Tubing (Spectrum Labs) against 300 mL TSM buffer (10 mM Tris-HCl, 100 mM sodium chloride, 1 mM magnesium chloride, pH 7.0) overnight with 2 buffer exchanges.

Sucrose Step-Gradient Velocity Sedimentation. The dialyzed cell-free reaction product was subjected to sucrose discontinuous gradient centrifugation. Polyallose 16×102 mm Centrifuge Tubes (Beckman, Palo Alto, Calif.) were successively filled with 1 mL each of sucrose solution decreasing by 2.5% w/v (40%, 37.5%, 35%, 32.5%, 30%, 27.5%, 25%, 22.5%, 20%, 17.5%, 15%, 12.5%, 10% w/v) in TSM buffer. The dialyzed cell-free reaction product was layered on top of the tube and centrifugation was performed at 31,000 rpm in a Beckman-Coulter SW-32 swinging bucket rotor (Fullerton, Calif.) in a Beckman L8-M ultracentrifuge at 4° C. for 3.5 hr with “slow” acceleration (profile 9) and “no brake” deceleration. The 0.5 mL fractions were collected using a Teledyne Isco Foxy Jr. Density Gradient Fractionation System (Lincoln, Nebr.) and the MS2 coat protein concentration in each fraction was determined by TCA-precipitation and radioactivity measurement using a liquid scintillation counter (LS3801, Beckman Coulter, Inc.)

Sucrose Gradient Fraction Radiolabeled MS2 Coat Protein Yield Determination. MS2 coat protein yields in each sucrose gradient fraction were determined by radioactivity measurements after 50 μl of each fraction was spotted on individual chromatography papers (Whatman, USA) and allowed to dry. The chromatography papers were each immersed in 5 mL of Beckman Readysafe Scintillation Cocktail and radiation was counted in a liquid scintillation counter (LS3801, Beckman Coulter, Inc.). The radiation was used to calculate the protein yield based on the molecular weight of the MS2 coat protein and the number of leucines in the MS2 coat protein.

VLP Concentration. Sucrose gradient factions containing VLPs were concentrated by filling Amicon Ultra-4 100,000 MWCO Centrifugal Filter Devices with gradient fractions and TSM buffer to 4 mL. The units were centrifuged for 15 min at 5,500 rpm and 4° C. in a Sorvall RC5B Centrifuge with a Fiberlite F13-14×15cy rotor (Piramoon Tech.) and Fiberlight 15 mL adaptors (Piramoon). The concentrated sample was immediately removed and stored at 4° C.

SDS-PAGE and Autoradiography. Soluble proteins were separated from insoluble fractions by centrifugation of samples at 15,000 g for 15 min. Five μl of sample was applied to a NuPAGE 10% Bis-Tris Gel (Invitrogen, La Jolla, Calif.) with Mark12 MW Standard (Invitrogen) molecular weight markers. Samples were run under reduced conditions in the presence of 62.5 mM dithiothreitol (DTT) (Invitrogen) and 7.25 μl NuPAGE LDS Sample Buffer (Invitrogen). Gels were stained with Commassie Blue Stain (BioRad) and dried with a gel dryer, model 583 (Bio-Rad, Richmond, Calif.) before being exposed to Kodak scientific imaging films (Rochester, N.Y.).

Transmission Electron Microscopy. 5 μl of 37 nM VLP solution (concentrated per methods) was applied to a carbon coated copper/Formvar grid and negatively stained with 1% w/v uranyl acetate, pH 4. Photographs were taken with a Gatan 967 CCD camera in a JEOL 1230 electron microscope at 80 kV acceleration voltage.

### Results

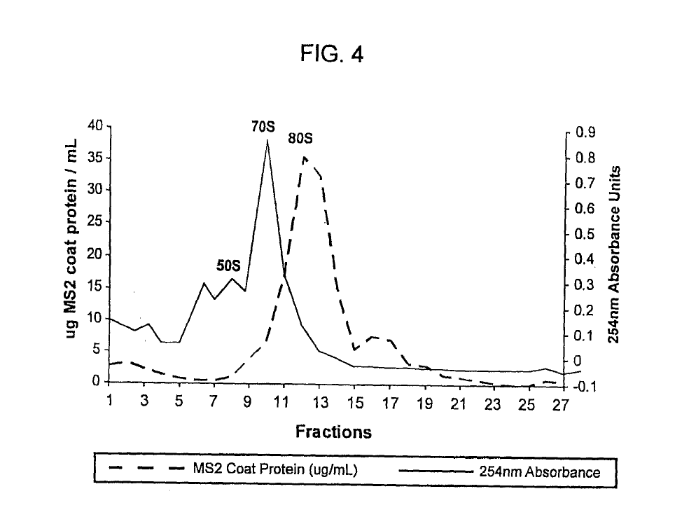

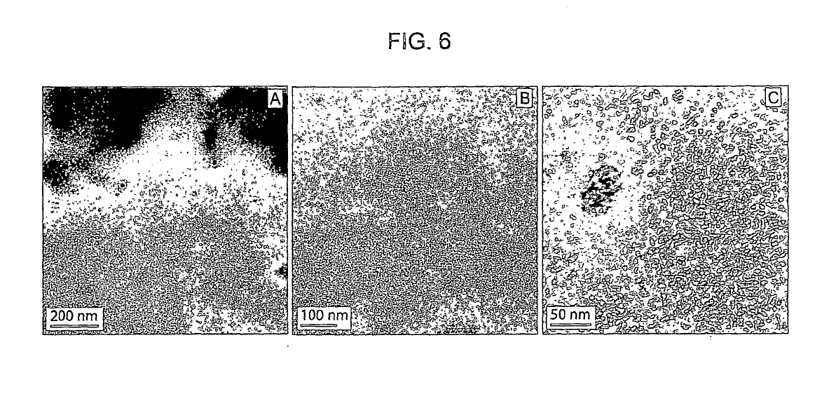

The assembly yield of MS2 coat protein expressed by the pET-MS2 cp vector in the prokaryote based cell-free reaction described in the methods section and determined by sucrose gradient velocity sedimentation followed by radiation counting was 295 μg/ml (+/−16 ug/ml, n=2) as indicated in FIG. 4. Fractions 9 though 15 of FIG. 4 were considered to contain properly assembled MS2 VLP. Based on the 254 nm absorbance profile which has peaks corresponding to the 50S and 70S ribosome complexes, the peak in MS2 coat protein VLP occurs at the expected 80S position. Since, the average total yield was 479 μg/ml (±16 ug/ml, n=5), the total assembly is approximately 62%. Accounting for losses due to concentration and transfer, we estimate that 73% of the expressed MS2 coat protein assembled into VLPs (FIG. 3 lane 3R indicates the concentration loss after dialysis). The actual production and isolation of MS2 VLPs was confirmed by TEM imaging of VLPs concentrated from fractions 12-15 (FIG. 6).

### Example 4

### Use of EDTA to Enhance Gradient Purification Technique

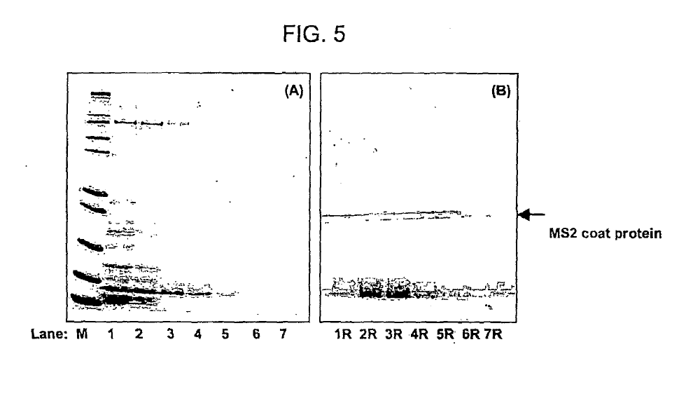

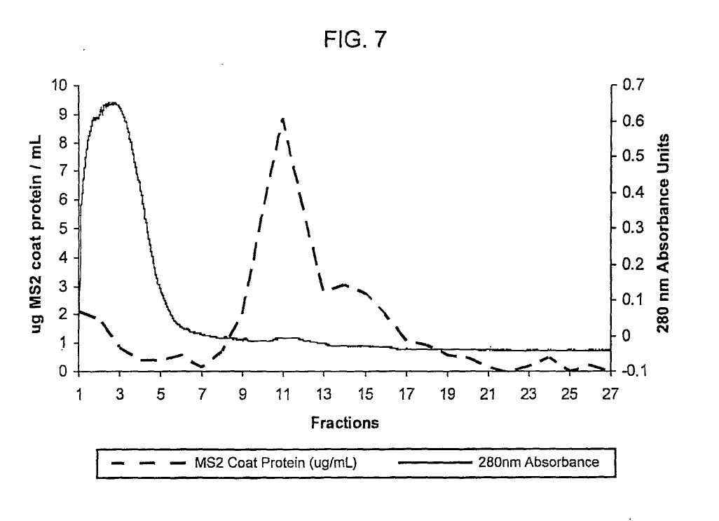

The same procedure was followed as in example 3, with the exception of adding 15 mM EDTA to the sucrose gradient. As shown by the 280 nm absorbance, the 15 mM EDTA dissociates the ribosome complexes (FIG. 7) thus decreasing the ribosome protein impurities in the MS2 VLP fractions as shown in FIG. 5A. Capsid assembly stability appeared to be unaffected by the 15 mM EDTA as the assembly yield was 57% (70% after accounting for transfer losses) as shown in FIG. 7.

### Example 5

### Cell-Free Produced MS2 Capsid pH Stability

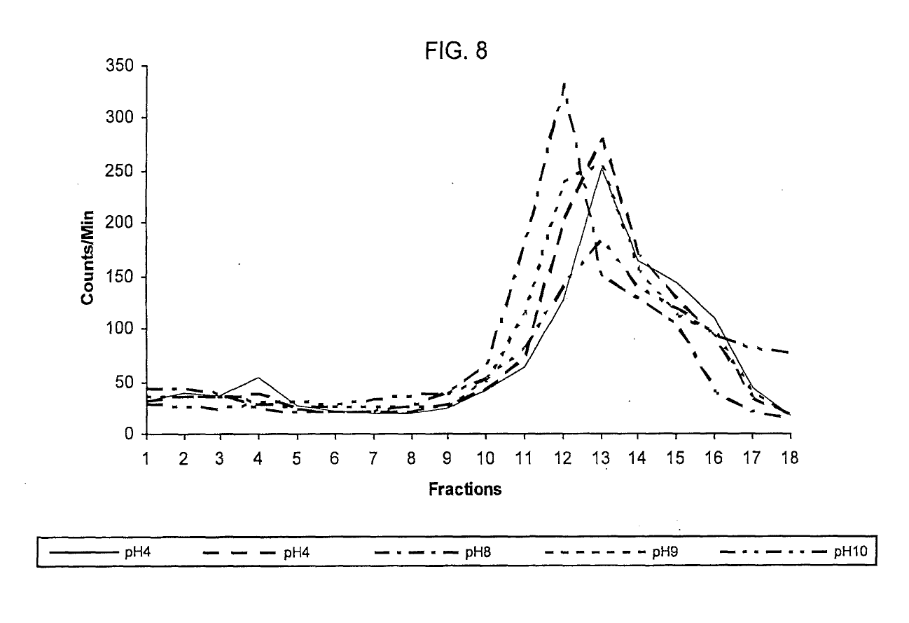

Following the methods of example 2, 100 μl of TSM buffer dialyzed PANOxSP cell-free reaction (with pET24a-MS2 cp) was incubated in 500 μl of TSM buffer adjusted to pH 4, 5, 8, 9 or 10 for 1 hr. A Sucrose Step-Gradient Velocity Sedimentation was performed and the radioactivity in the fractions was counted following the methods of example 3, with the MS2 coat protein concentration shown in FIG. 8 in terms of radioactivity (counts/min) instead of MS2 coat protein concentration (ug/ul). The capsid appears to be stable at pH 4 through 9, and moderately stable at pH 10, which is in substantial agreement to the pH stability observed for in vivo produced viral capsids (Hooker et al. 2004 J Am Chem Soc 126:3718-3719).

### REFERENCES

Davanloo P, Rosenberg A H, Dunn J J, Studier F W. 1984. Cloning and expression of the gene for bacteriophage T7RNApolymerase. Proc Natl Acad Sci USA 81(7):2035-2039.
Klein et al. 2004. Unique Features of Hepatitis C Virus Capsid Formation Revealed by De Novo Cell-Free Assembly. Journal of Virology 78(17): 9257-9269.
Lingappa et al. 2005. Comparing capsid assembly of primate lentiviruses and hepatitis B virus using cell-free systems. Virology 333:114-123.
Katanaev et al. 1996. Formation of bacteriophage MS2 infectious units in a cell-free translation system. FEBS 397:143-148.
Spirin et al. 1988. A continuous cell-free translation system capable of producing polypeptides in high yield. Science 242:1162-1164

### Example 6

### Hepatitis B Core Antigen VLP Synthesis

Hepatitis B plasmid construction. The human Hepatitis B core antigen of subtype adyw sequence (Pasek et al. 1979 Nature 282(5739):575-579) with the C-terminus truncated at amino acid 149 was optimized for E. coli tRNA concentrations and was synthesized from oligonucleotides designed with DNAworks v3.0. The vector pET24a-HBc149 was prepared using the same procedure as for pET24a-MS2 cp with the exception of using Xho I instead of the Sal I restriction site.

Expression of Hepatitis B core antigen (monomer of Hepatitis B VLP) and MS2 coat protein. The MS2 core protein and Hepatitis B core antigen were produced in 30 μL reactions as described above at 479 μg/mL (SD=16 μg/mL, n=3) and 445 μg/mL (SD=18 μg/mL, n=3) and over 96% was soluble. The MS2 core protein and Hepatitis B core antigen were produced at comparable yields of 525 μg/mL (SD=25 μg/mL, n=3) and 436 μg/mL (SD=13 μg/mL, n=3) and over 92% was soluble.

Demonstrating Assembly of MS2 Capsid and Hepatitis B VLP. The assembly efficiency for both the MS2 VLP and the HBV VLP in 30 μL reaction volumes was 80% or greater resulting in assembly yields of 384 μg/mL (SD=32 μg/mL, n=3) and 356 μg/mL respectively (n=1). The VLP assembly yields and efficiencies at 1 mL reaction volumes were also at 80% or greater efficiency with VLP yields of 442 μg/mL (SD=16 μg/mL, n=3) and 372 μg/mL (SD=14 μg/mL, n=1) for the MS2 and HBV VLPs.

pH Stability. Cell-free polypeptide synthesis (CFPS) synthesized VLPs also demonstrated pH stabilities slightly better or comparable to in vivo produced VLPs. Purified VLPs were incubated for 12 hours at pHs ranging from 3 to 12 and analyzed by 10%-40% continuous sucrose density velocity sedimentation. Remarkably the CFPS synthesized MS2 VLP demonstrated stability over the pH range of 3 through 11 with 78% or greater recovery efficiency. Hooker et al. (2004) Journal of the American Chemical Society 126(12):3718-3719 reported 75% or greater recovery over pH 3 through 10 after a similar 12 hour incubation with in vivo produced MS2 VLPs. The CFPS produced HBV VLP demonstrated greater than 67% recovery of the VLPs after a 12 hour incubation at pHs 5 through 10. Similarly, recombinant Hepatitis B core antigen VLPs produced in Escherichia coli have been reported to be unstable at pHs less than 5.0 and greater than 10.5 after 90 minutes (Nath et al. 1992 Journal of Clinical Microbiology 30(6):1617-1619).

Characterization by TEM—Transmission Electron Microscopy with 1% w/v uranyl acetate negative staining was used to verify that the peak fractions of MS2 coat protein and Hepatitis B core antigen from the sucrose gradient contained correctly formed VLPs. The average diameter was 27.2 nm (SD=0.9 nm, n=100) for MS2 VLP and 30.7 nm (SD=1.3 nm, n=100) for HBV VLP which agrees with reported diameters of 27 nm for MS2 VLP and 30 nm for full-length HBV VLP produced using in vivo methods (Hooker et al. 2004; Zlotnick et al. 1996 Biochemistry 35(23):7412-7421).

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

What is claimed is:

1. A method for synthesis of virus like particles in a cell-free in vitro reaction, the method comprising:
synthesizing virus coat proteins in a prokaryotic cell-free in vitro translation reaction substantially free of polyethylene glycol and comprising a bacterial cell extract, components of polypeptide and/or mRNA synthesis machinery; a template for transcription of the polypeptide; monomers for synthesis of the polypeptide; and co-factors, enzymes and other reagents necessary for translation;
wherein the virus coat proteins self-assemble into a stable virus like particle free of a viral genome, and comprising at least 60 separate proteins.

2. The method according to claim 1, wherein said reaction mixture produces at least about 250 μg/ml of virus coat protein.

3. The method according to claim 2, wherein at least about 50% of said coat protein is assembled into virus like particles.

4. The method according to claim 3, wherein said virus like particle comprises one species of coat protein.

5. The method according to claim 3, wherein said virus like particle comprises two or more species of coat protein.

6. The method of claim 1, wherein the virus like particle has an icosahedral geometry.

7. The method of claim 1, wherein said virus coat protein is a bacteriophage coat protein.

8. The method of claim 7, wherein said bacteriophage is MS2.
a stop codon.

9. The method according to claim 1, wherein oxidative phosphorylation is activated in the cell-free in vitro translation reaction.

10. A kit for use in any of methods according to claims 1-9.


SEQUENCE LISTING (1 sequence)

SEQ ID NO: 1 - DNA, 405 bp, Artificial Sequence
  Synthetic polynucleotide
catatggccagcaactttacccagttcgttctggttgacaacggcggtacgggcgacgtgacggtggcgccgtctaacttcgccaacggtgttgcggagtggatttcttctaattctcgcagccaagcctataaagttacgtgttctgtgcgtcagtctagcgcccagaatcgcaagtacaccattaaagtggaggtgccgaaggtggcgacgcaaaccgtgggtggtgtggagctgccagttgcggcctggcgtagctatctgaacatggagctgacgatcccaatctttgccaccaatagcgactgcgaactgattgtgaaggccatgcagggtctgctgaaggatggtaacccgattccatctgccattgccgcgaactctggtatctactaataagtcgac